(19) United States
(12) Patent Application Publication
(10) Pub. No.: US 2023/0307219 A1
(43) Pub. Date: Sep. 28, 2023

(54) MASS SPECTROMETRY METHOD AND MASS SPECTROMETER

(71) Applicant: HITACHI HIGH-TECH CORPORATION, Tokyo (JP)

(72) Inventors: Ryusuke HIROSE, Tokyo (JP); Hideyuki AKIYAMA, Tokyo (JP); Noriaki SAKAI, Tokyo (JP)

(21) Appl. No.: 18/013,060

(22) PCT Filed: Jul. 17, 2020
(86) PCT No.: PCT/JP2020/027826

§ 371 Date: Dec. 27, 2022

(51) Int. Cl. H01J 49/00; G01N 27/623; H01J 49/26

(52) U.S. Cl. CPC H01J 49/0036; G01N 27/623; H01J 49/0031; H01J 49/26

(57) ABSTRACT

A mass spectrometry method calculates a theoretical mass spectrum by calculating, on the basis of a molecular formula of a component to be analyzed and an isotope abundance ratio of an element that is included in the component to be analyzed and for which a plurality of isotopes exists, the mass of the isotopes and the abundance ratio of the component to be analyzed for each mass. A measurement target is ionized and the mass of the ionized ions and a number of ions in each mass is detected. A first mass spectrum is calculated based on the detection result; and a degree of matching is calculated by comparing the theoretical mass spectrum and the first mass spectrum, for only the mass in which a peak of the theoretical mass spectrum exists. On the basis of the degree of matching, the presence/absence of the component to be analyzed is determined.

### TECHNICAL FIELD

The present disclosure relates to a mass spectrometry method and a mass spectrometer.

### BACKGROUND ART

When a measurement target is analyzed by a mass spectrometer, it is necessary to determine whether a peak of an obtained mass spectrum is derived from a component to be analyzed or from a factor other than the component to be analyzed. In particular, when isotopes or homologs exist in the component to be analyzed, the number of peaks of the mass spectrum increases. For example, a large number of components having different numbers of carbon atoms and chlorine atoms exist in a chlorinated paraffin, and the number of peaks may reach several hundred in a mass spectrum obtained by measurement.

In general, an isotope abundance ratio is known, and a result reflecting the isotope abundance ratio is obtained also in the mass spectrum. PTL 1 discloses a technique for performing qualitative analysis of a sample on the basis of an isotope abundance ratio of a component to be analyzed.

### CITATION LIST

### Patent Literature

PTL 1: JP-A-2010-66036

### SUMMARY OF INVENTION

### Technical Problem

However, when there is a peak caused by a factor other than the component to be analyzed, since the peak is also analyzed, an erroneous determination is caused in qualitative analysis.

Therefore, the disclosure provides a technique for easily avoiding an influence of a component other than the component to be analyzed in mass spectrometry.

### Solution to Problem

In order to achieve the above object, a mass spectrometry method of the disclosure is a mass spectrometry method using a mass spectrometer, the mass spectrometry method includes: calculating, by a control unit of the mass spectrometer, a theoretical mass spectrum by calculating, on the basis of a molecular formula of a component to be analyzed and an isotope abundance ratio of an element that is included in the component to be analyzed and for which a plurality of isotopes exist, a mass of the isotopes of the component to be analyzed and an abundance ratio of the component to be analyzed for each mass; ionizing, by a preprocessing unit of the mass spectrometer, a measurement target; detecting, by a mass detection unit of the mass spectrometer, a mass of ionized ions and the number of ions in each mass; calculating, by the control unit, a first mass spectrum on the basis of a detection result of the mass detection unit; calculating, by the control unit, a degree of matching by comparing the theoretical mass spectrum and the first mass spectrum, for only a mass in which a peak of the theoretical mass spectrum exists; and determining, by the control unit and on the basis of the degree of matching, presence or absence of the component to be analyzed in the measurement target.

Further features related to the disclosure will become apparent from a description of the present specification and the accompanying drawings. In addition, aspects of the disclosure may be achieved and implemented using elements, combinations of various elements, the following detailed description, and accompanying claims.

The description of the present specification is merely exemplary, and is not intended to limit the scope of claims or application examples of the disclosure in any sense.

### Advantageous Effects of Invention

According to the technique of the disclosure, an influence other than the component to be analyzed can be easily avoided.

Problems, configurations, and effects other than those described above will become apparent from the following description of the embodiments.

### BRIEF DESCRIPTION OF DRAWINGS

FIG. 1 is a functional configuration diagram of a mass spectrometer according to a first embodiment.

FIG. 2 is a flowchart of a mass spectrometry method according to the first embodiment.

FIG. 3 is a diagram illustrating an example of a theoretical mass spectrum.

FIG. 4 is a diagram illustrating an example of a peak of a mass spectrum obtained by measurement.

FIG. 5 is a flowchart of a mass spectrometry method according to a second embodiment.

FIG. 6 is a flowchart of a mass spectrometry method according to a third embodiment.

FIG. 7 is a conceptual diagram of a method for separating an ion intensity.

FIG. 8 is a flowchart of a mass spectrometry method according to a fourth embodiment.

### DESCRIPTION OF EMBODIMENTS

### First Embodiment

### Configuration Example of Mass Spectrometer

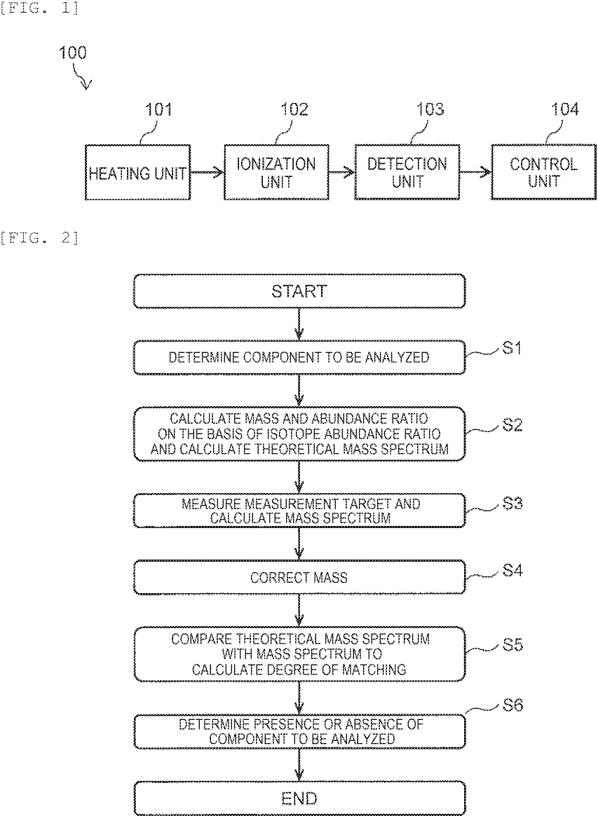

FIG. 1 is a functional configuration diagram of a mass spectrometer 100 according to a first embodiment. In the present embodiment, a case in which the mass spectrometer 100 is a thermal desorption mass spectrometer will be described as an example. The thermal desorption mass spectrometer heats a measurement target (sample) to generate a gas component, ionizes the gas component, and performs mass spectrometry on the gas component. A mass spectrometer to which the technique of the disclosure can be applied is not limited to the thermal desorption mass spectrometer, and the technique of the disclosure can be applied to a gas chromatograph mass spectrometer or a liquid chromatograph mass spectrometer that separates a compound in a measurement target by a separation column.

As illustrated in FIG. 1, the mass spectrometer 100 includes a heating unit 101 (preprocessing unit), an ionization unit 102 (preprocessing unit), a detection unit 103 (mass detection unit), and a control unit 104 (calculation unit).

The heating unit 101 heats a measurement target to generate a gas component. The heating unit 101 can include, for example, a heating furnace, and the measurement target can be conveyed by an autosampler to a heating chamber of the heating furnace.

The ionization unit 102 can include a known ionization device, and ionizes the gas component generated by the heating unit 101. Examples of an ionization method of the ionization unit 102 include atmospheric pressure chemical ionization (APCI) method, electrospray ionization (ESI) method, atmospheric pressure photoionization (APPI) method, and electron ionization (EI) method. Among them, atmospheric pressure chemical ionization method is less likely to cause destruction of a structure of a component to be analyzed in ionization (fragmentation of gas components) and is less likely to cause a fragment peak, and thus can detect the component to be analyzed without separation by a chromatograph or the like.

The detection unit 103 can include a known mass analyzer, and detects a mass of ions ionized by the ionization unit 102 and the number of ions (ion intensities) for each mass. The detection unit 103 outputs a detection signal of the ion intensities to the control unit 104. In addition, the detection unit 103 may output an ion current to the control unit 104 as a detection signal.

The control unit 104 calculates a mass spectrum on the basis of the mass of the ions and the number of ions for each mass detected by the detection unit 103, and analyzes the measurement target. In addition, the control unit 104 controls the overall operation of the mass spectrometer 100. The control unit 104 can include, for example, a memory in which a program for operating each unit of the mass spectrometer 100 is stored and a processor (CPU, MPU, or the like) that executes the program. The control unit 104 can be incorporated into a computer terminal such as a personal computer or a smartphone, and the control unit 104 is connected to, a storage device that stores various data, an input device for a user to input an instruction to the mass spectrometer 100, a display device that displays a mass spectrometry result or various GUI screens, or the like.

### Mass Spectrometry Method

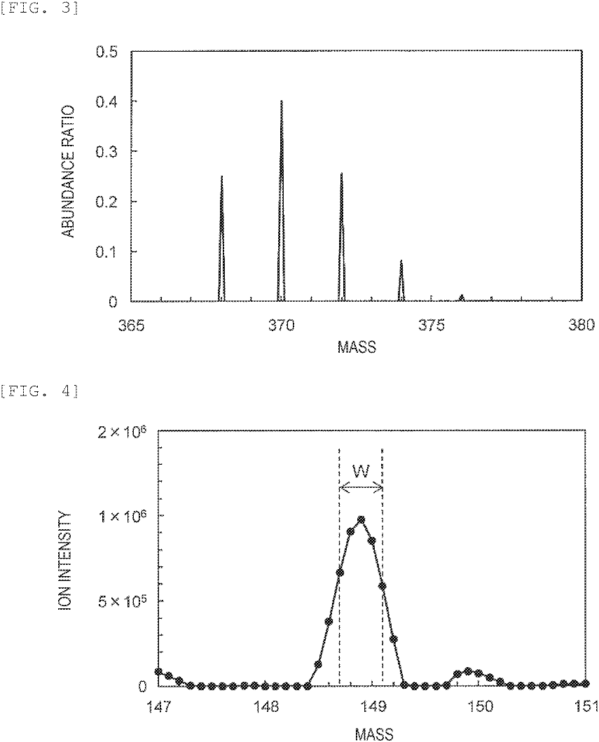

FIG. 2 is a flowchart illustrating a mass spectrometry method according to the first embodiment.

### Step S1

The user of the mass spectrometer 100 determines a component to be analyzed. Specifically, the control unit 104 displays a GUI screen for the user to determine the component to be analyzed on the display device, and the user uses the input device to input a desired component to be analyzed via the GUI screen. Here, data on a compound that can be the component to be analyzed may be stored as a database, or the user may select a compound from the database. In addition, the user may input a chemical formula (molecular formula, structural formula, or the like) of the component to be analyzed. Information of the input component to be analyzed is output to the control unit 104. In the present embodiment, a case in which the component to be analyzed is a chlorinated paraffin will be described as an example.

### Step S2

The control unit 104 calculates, on the basis of the molecular formula of the component to be analyzed and an isotope abundance ratio, a mass of each isotope and an abundance ratio, and calculates a theoretical mass spectrum. The molecular formula of the compound that can be the component to be analyzed and the isotope abundance ratio may be stored as a database and read by the control unit 104, or may be calculated by the control unit 104 according to the chemical formula of the component to be analyzed input by the user.

The chlorinated paraffin is a generic term for a compound in which chlorine is bonded to an alkane (molecular formula: CₙH₂ₙ₊₁), and is a mixture of components having different numbers of carbon atoms and chlorine atoms. Therefore, a molecular formula of the chlorinated paraffin is represented by CₓH_{2x+2-y}Cl_{y}, where x represents the number of carbon atoms and y represents the number of chlorine atoms. Chlorine has two stable isotopes, i.e., ³⁵Cl and ³⁷Cl, and isotope abundance ratios thereof are ³⁵Cl (75.77%) and ³⁷C1 (24.33%). Therefore, even if x and y are the same, y + 1 types of isotopes having different masses exist.

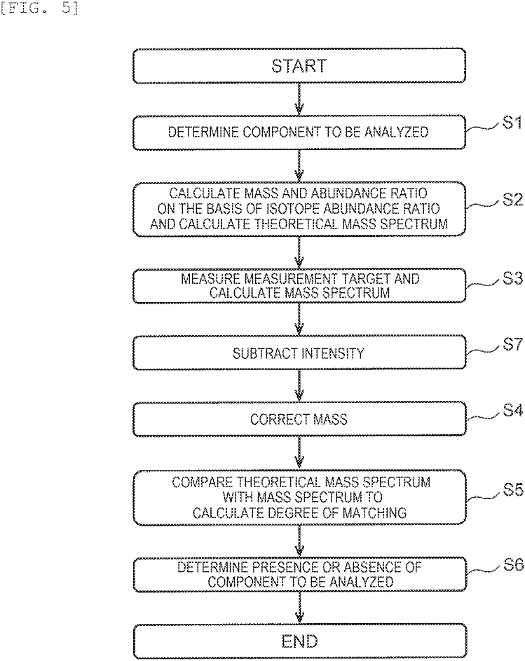

FIG. 3 is a theoretical mass spectrum calculated from the mass and the abundance ratio calculated for x = 14 and y = 5. A horizontal axis represents the mass, a vertical axis represents the abundance ratio calculated on the basis of the isotope abundance ratio of chlorine, and a total value of six peaks is calculated to be 1. The theoretical mass spectrum can be calculated by performing the same calculation on all combinations of x and y to be analyzed.

### Step S3

Returning to FIG. 2, the control unit 104 measures a measurement target suspected of including the component to be analyzed by the mass spectrometer 100, and acquires a mass spectrum (hereinafter, may be referred to as a “first mass spectrum”). Specifically, the control unit 104 drives the heating unit 101 and the ionization unit 102 to gasify and ionize the component to be analyzed, and receives an input of the detection signal of the detection unit 103. The control unit 104 obtains the mass spectrum with the mass (mass-to-charge ratio m/z) as a horizontal axis and the ion intensity as a vertical axis.

### Step S4

The control unit 104 corrects a mass of the mass spectrum acquired in step S3 in accordance with an ionization reaction in the ionization unit 102. For example, when oxygen ions (O₂⁻) are added in the ionization, the control unit 104 shifts the horizontal axis of the obtained mass spectrum to a minus side by 32 Da. That is, the control unit 104 moves a peak at 448 Da on the horizontal axis to 416 Da.

### Step S5

The control unit 104 compares the theoretical mass spectrum calculated in step S2 with the mass spectrum acquired in step S3 to calculate a degree of matching. In the calculation of the degree of matching, a peak that is a comparison target is only a mass in which a peak of the theoretical mass spectrum exists, and the other peaks are not the comparison target. Accordingly, even if there is a peak other than the component to be analyzed in the mass spectrum acquired in step S3, the peak can be excluded from the comparison target. Here, the peak other than the component to be analyzed is caused by the mass spectrometer 100 itself, a container of the measurement target, impurity components other than the component to be analyzed included in the measurement target, and the like.

There are y + 1 types of peaks in the theoretical mass spectrum of the chlorinated paraffin, among which the peak that is the comparison target is not limited. For example, all peaks may be the comparison target, a plurality of peaks having strong intensities among peaks of the mass spectrum acquired in step S3 may be the comparison target, or peaks exceeding a predetermined threshold may be the comparison target.

A method of calculating the degree of matching is not particularly limited, and, for example, a correlation coefficient can be used.

FIG. 4 is a diagram illustrating an example of a peak of a mass spectrum obtained by mass spectrometry. As illustrated in FIG. 4, in general, each peak of the mass spectrum obtained by the mass spectrometer is not a single line but spreads to some extent, for example, spreads with a Gaussian function. Depending on the measurement, the spread may change with time, or peak positions may shift. This makes a calculated value of the degree of matching unstable. Therefore, the control unit 104 can stabilize the value of the degree of matching by calculating, with respect to the peak of the mass spectrum obtained in step S3, an average value of the ion intensities within a range of a predetermined width W on the horizontal axis and using the average value for the calculation of the degree of matching.

### Step S6

Returning to FIG. 2, the control unit 104 determines presence or absence of the component to be analyzed on the basis of the degree of matching. A determination method is not limited, for example, the control unit 104 can determine that the component to be analyzed is “present” when the degree of matching is equal to or larger than a threshold set in advance, and determine that the component to be analyzed is “absent” when the degree of matching is smaller than the threshold. Alternatively, the control unit 104 may set a first threshold and a second threshold larger than the first threshold in advance, determine that the component to be analyzed is “absent” when the degree of matching is equal to or smaller than the first threshold, and determine that the component to be analyzed is “present” when the degree of matching is equal to or larger than the second threshold.

### Summary of First Embodiment

As described above, in the first embodiment, the degree of matching between the mass spectrum obtained by the measurement and the theoretical mass spectrum is calculated only for the mass in which the peak of the theoretical mass spectrum of the component to be analyzed exists, and the presence or absence of the component to be analyzed is determined on the basis of the degree of matching. Accordingly, in the mass spectrum obtained by the measurement, even if there is a peak in a mass other than the component to be analyzed, the peak is not used for the calculation of the degree of matching. Therefore, since an influence of the peak other than the component to be analyzed can be avoided, an erroneous determination in qualitative analysis can be prevented.

The mass spectrometry method according to the present embodiment is not limited to the above analysis of the chlorinated paraffin, and can be applied to analysis of a compound containing an element in which a plurality of stable isotopes exist and the isotope abundance ratio is not negligible. In the mass spectrometry method according to the present embodiment, for example, an organic compound such as an organic halogen compound or an organic metal compound may be used as the component to be analyzed. As the organic halogen compound, organic chlorine compounds such as chlorinated paraffins or dioxins, and organic bromine compounds such as bromine-based flame retardants (for example, tetrabromobisphenol A) or brominated dioxins can be used as the component to be analyzed.

### Second Embodiment

In the first embodiment, a method of calculating the degree of matching by calculating the theoretical mass spectrum of the component to be analyzed and the mass spectrum obtained by measuring the measurement target is described. The mass spectrum obtained by measuring the measurement target includes not only the compound contained in the measurement target, but also the peak caused by an element other than the measurement target such as the mass spectrometer 100 itself or the container of the measurement target. When the element other than the measurement target has the same mass as the component to be analyzed, that is, when a peak of the element other than the measurement target is a peak at the same position as the component to be analyzed, an adverse influence (erroneous determination) occurs in the measurement. Therefore, in the second embodiment, a technique for reducing an influence of the element other than the measurement target is proposed.

A mass spectrometer according to the present embodiment can be the same as the mass spectrometer 100 described in the first embodiment.

### Mass Spectrometry Method

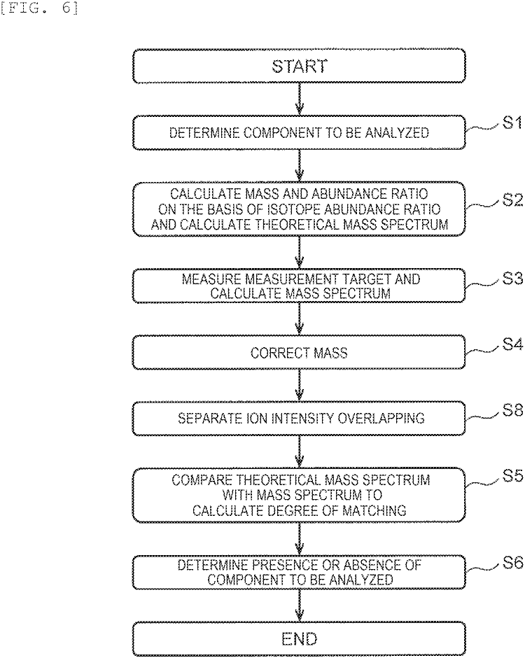

FIG. 5 is a flowchart of a mass spectrometry method according to the second embodiment. Steps S1 to S6 are the same as those in the first embodiment, and thus the description thereof will be omitted. In the present embodiment, step S7 is performed before step S5. In step S7, the control unit 104 subtracts a mass spectrum (hereinafter, may be referred to as a “second mass spectrum”) measured in advance by operating the mass spectrometer 100 in a state where the measurement target is absent from the mass spectrum (first mass spectrum) obtained in step S3. Specifically, the control unit 104 subtracts an ion intensity of a peak of the second mass spectrum from the ion intensity of the peak of the first mass spectrum. Here, as described with reference to FIG. 3, the calculation of this step can be performed with the average value of the ion intensities at the predetermined width W of each peak as the ion intensity of the peak. The second mass spectrum includes only the peak caused by the element other than the measurement target such as the mass spectrometer 100 or the container of the measurement target. Therefore, the mass spectrum of only the measurement target can be obtained by the processing of step S7. By comparing the mass spectrum with the theoretical mass spectrum, erroneous determination can be prevented.

Step S7 is only required to be performed between step S3 and step S5, and an order of step S4 and step S7 is not limited. In addition, the acquisition of the mass spectrum in the state where the measurement target is absent can be performed before measuring the measurement target in step S3.

### Summary of Second Embodiment

As described above, in the second embodiment, the second mass spectrum acquired in the state where the measurement target is absent is subtracted from the first mass spectrum obtained by measuring the measurement target, and the degree of matching between the mass spectrum obtained by the subtraction and the theoretical mass spectrum of the component to be analyzed is calculated. Accordingly, since the influence of the peak caused by the elements other than the measurement target can be eliminated, analysis accuracy can be further improved as compared with the first embodiment.

### Third Embodiment

In the first and second embodiments, a technique for analyzing in consideration of the isotope abundance ratio of the element in the component to be analyzed is described. Depending on the component to be analyzed, peak positions may overlap among homologs, which may lead to erroneous determination if peak intensities of the homologs are not separated from each other. Therefore, in the third embodiment, a technique for separating the peaks overlapping among the homologs is proposed.

A mass spectrometer according to the present embodiment can be the same as the mass spectrometer 100 described in the first embodiment.

### Mass Spectrometry Method

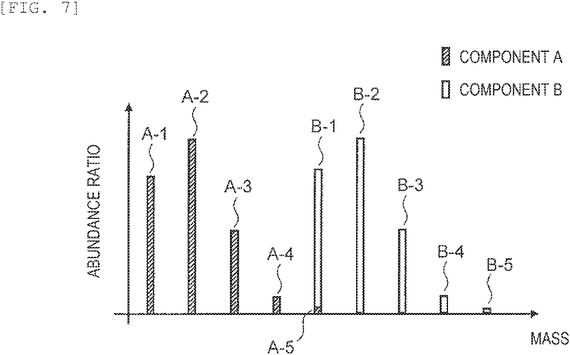

FIG. 6 is a flowchart of a mass spectrometry method according to the third embodiment. Steps S1 to S6 are the same as those in the first embodiment, and thus the description thereof will be omitted. In the present embodiment, step S8 is performed before step S5. In step S8, the control unit 104 separates the ion intensities of the peaks of the mass spectrum obtained in step S3 when there is a peak at a position having the same mass between components (homologs) in which combinations of the number of carbon atoms x and the number of chlorine atoms y are different in the theoretical mass spectrum. An example of a method for separating the ion intensity (overlapping of peaks) will be described below.

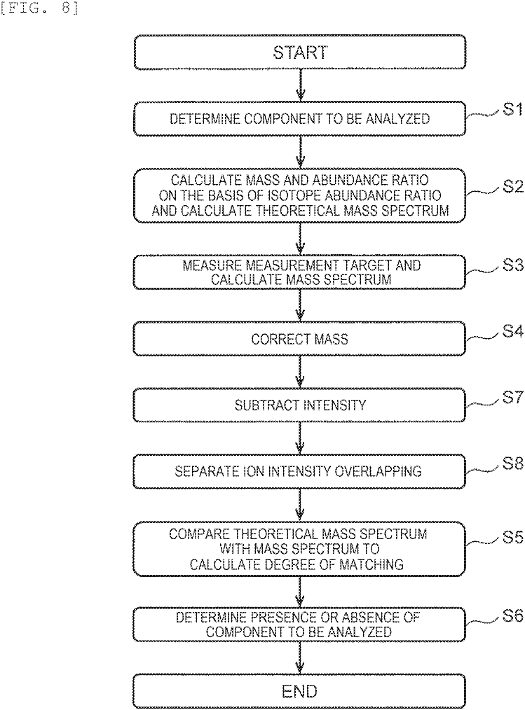

FIG. 7 is a conceptual diagram of the method for separating the ion intensity. Here, a component A and a component B (chlorinated paraffin) having different combinations of the number of carbon atoms x and the number of chlorine atoms y will be described as examples. FIG. 7 illustrates mass spectra calculated from masses and abundance ratios of the component A (component A-1 to component A-5) and the component B (component B-1 to component B-5), and five peaks appear respectively. The number following the letter of each component is the number of ³⁷Cl contained in each component. Therefore, when the number increases by 1, the mass increases by 2. In the example of FIG. 7, it is assumed that the masses of the component A-5 and the component B-1 are the same, that is, the peak positions overlap each other. In this case, the abundance ratios (vertical axis) of the non-overlapping peaks (component A-2) and the overlapping peaks (component A-5) can be calculated from the theoretical mass spectrum. Therefore, the ion intensity of the component A-5 of the mass spectrum obtained in step S3 can be calculated on the basis of the ion intensity of the component A-2 and the above ratio. The ion intensity of the component B-1 can also be acquired from a calculation result of the ion intensity of the component A-5. In this manner, even if there is a peak having the same mass between the component A and the component B, the ion intensities can be separated. Here, as described with reference to FIG. 3, the average value of the ion intensities in the predetermined width W of each peak can be used for the calculation of the ratio of the present step as the ion intensity of the peak.

The peak used in the processing of the present step (non-overlapping peaks between the homologs) is not limited, and a peak having the largest ion intensity may be used, or a plurality of peaks may be used. In addition, Step S8 is only required to be performed between step S3 and step S5, and an order of step S4 and step S8 is not limited.

### Summary of Third Embodiment

As described above, in the third embodiment, when there are components having the same mass between the homologs having different combinations of the number of carbon atoms x and the number of chlorine atoms y, the ion intensities of the components having the same mass are separated from the mass spectrum obtained by measuring the measurement target. Accordingly, even if homologs having the same mass (the peaks are at the same position) exist, since each homolog can be compared with the theoretical mass spectrum, an erroneous determination is prevented.

### Fourth Embodiment

In the fourth embodiment, a combination of the second embodiment and the third embodiment will be described.

FIG. 8 is a flowchart of a mass spectrometry method according to the fourth embodiment. Contents of each processing of steps S1 to S8 are as described above. As illustrated in FIG. 8, step S7 and step S8 can be performed between step S4 and step S5. Step S7 is only required to be performed between step S3 and step S5, and may be performed before step S4, or may be performed after step S8.

### Modification

The disclosure is not limited to the above embodiments, and includes various modifications. For example, the above embodiments have been described in detail for easy understanding of the disclosure, and are not necessarily limited to those including all the configurations described above. A part of the configurations in one embodiment can be replaced with a configuration in another configuration. The configuration of another embodiment may be added to the configuration of one embodiment. A part of the configuration of each embodiment may be added, deleted, or replaced with a part of the configuration of another embodiment.

Reference Signs List

| | |
|---|---|
| 100: | mass spectrometer |
| 101: | heating unit |
| 102: | ionization unit |
| 103: | detection Unit |
| 104: | control Unit |

1. A mass spectrometry method using a mass spectrometer, the mass spectrometry method comprising:
calculating, by a control unit of the mass spectrometer, a theoretical mass spectrum by calculating, on the basis of a molecular formula of a component to be analyzed and an isotope abundance ratio of an element that is included in the component to be analyzed and for which a plurality of isotopes exist, a mass of the isotopes of the component to be analyzed and an abundance ratio of the component to be analyzed for each mass;
ionizing, by a preprocessing unit of the mass spectrometer, a measurement target;
detecting, by a mass detection unit of the mass spectrometer, a mass of ionized ions and the number of ions in each mass;
calculating, by the control unit, a first mass spectrum on the basis of a detection result of the mass detection unit;
calculating, by the control unit, a degree of matching by comparing the theoretical mass spectrum and the first mass spectrum, for only a mass in which a peak of the theoretical mass spectrum exists; and
determining, by the control unit and on the basis of the degree of matching, presence or absence of the component to be analyzed in the measurement target.

2. The mass spectrometry method according to claim 1, wherein
the component to be analyzed is an organic halogen compound, and
in calculating the theoretical mass spectrum, the control unit calculates, on the basis of a molecular formula of the organic halogen compound and an isotope abundance ratio of halogen, masses of y + 1 types of isotopes of the organic halogen compound and an abundance ratio of the organic halogen compound for each mass, for each of combinations of the number of carbon atoms x and the number of halogen atoms y of the organic halogen compound.

3. The mass spectrometry method according to claim 1, further comprising:
operating, by the control unit, the preprocessing unit and the mass detection unit in a state where the measurement target is absent;
calculating, by the control unit, a second mass spectrum when the measurement target is absent on the basis of the detection result of the mass detection unit; and
subtracting, by the control unit, the second mass spectrum from the first mass spectrum before calculating the degree of matching.

4. The mass spectrometry method according to claim 1, further comprising:
separating, by the control unit, ion intensities of components having the same mass from the first mass spectrum when the components having the same mass are present between homologs of the component to be analyzed before calculating the degree of matching.

5. The mass spectrometry method according to claim 1, wherein
the control unit calculates the degree of matching on the basis of an average ion intensity within a range of a predetermined width of the first mass spectrum.

6. The mass spectrometry method according to claim 1, wherein
the control unit calculates the degree of matching by using a correlation coefficient of the number of ions of the mass in which the peak exists.

7. The mass spectrometry method according to claim 1, wherein
in the determination, the control unit compares the degree of matching with a threshold, and determines that the component to be analyzed is present when the degree of matching is equal to or larger than the threshold.

8. The mass spectrometry method according to claim 1, wherein
in the determination, the control unit compares the degree of matching with a first threshold and a second threshold larger than the first threshold, determines that the component to be analyzed is absent when the degree of matching is smaller than the first threshold, and determines that the component to be analyzed is present when the degree of matching is equal to or larger than the second threshold.

9. The mass spectrometry method according to claim 1, further comprising:
correcting, by the control unit, a mass of the first mass spectrum in accordance with an ionization reaction in the preprocessing unit.

10. The mass spectrometry method according to claim 1, wherein
the component to be analyzed is an organic chlorine compound.

11. The mass spectrometry method according to claim 1, wherein
the component to be analyzed is an organic bromine compound.

12. The mass spectrometry method according to claim 1, wherein
an ionization method of the preprocessing unit is atmospheric pressure chemical ionization method.

13. A mass spectrometer comprising:
a preprocessing unit configured to ionize a measurement target;
a mass detection unit configured to detect a mass of ions ionized by the preprocessing unit and the number of ions in each mass; and
a control unit configured to control the preprocessing unit and the mass detection unit, wherein
the control unit is configured to execute:
processing of calculating a theoretical mass spectrum by calculating, on the basis of a molecular formula of a component to be analyzed and an isotope abundance ratio of an element that is included in the component to be analyzed and for which a plurality of isotopes exist, a mass of the isotopes of the component to be analyzed and an abundance ratio of the component to be analyzed for each mass;
processing of calculating a first mass spectrum on the basis of a detection result of the mass detection unit;
processing of calculating a degree of matching by comparing the theoretical mass spectrum and the first mass spectrum, for only a mass in which a peak of the theoretical mass spectrum exists; and
processing of determining, on the basis of the degree of matching, presence or absence of the component to be analyzed in the measurement target.

14. The mass spectrometer according to claim 13, wherein
the component to be analyzed is an organic halogen compound, and
in the processing of calculating the theoretical mass spectrum, the control unit is configured to calculate, on the basis of a molecular formula of the organic halogen compound and an isotope abundance ratio of halogen, masses of y + 1 types of isotopes of the organic halogen compound and an abundance ratio of the organic halogen compound for each mass, for each of combinations of the number of carbon atoms x and the number of halogen atoms y of the organic halogen compound.

15. The mass spectrometer according to claim 13, wherein
the control unit is configured to further execute:
processing of operating the preprocessing unit and the mass detection unit in a state where the measurement target is absent;
processing of calculating a second mass spectrum when the measurement target is absent on the basis of the detection result of the mass detection unit; and
processing of subtracting the second mass spectrum from the first mass spectrum before calculating the degree of matching.

16. The mass spectrometer according to claim 13, wherein
the control unit is configured to further execute:
processing of separating ion intensities of components having the same mass from the first mass spectrum when the components having the same mass are present between homologs of the component to be analyzed before calculating the degree of matching.

17. The mass spectrometer according to claim 13, wherein
the control unit is configured to calculate the degree of matching on the basis of an average ion intensity within a range of a predetermined width of the first mass spectrum.

18.
The mass spectrometer according to claim 13, wherein
the control unit is configured to calculate the degree of matching by using a correlation coefficient of the number of ions of the mass in which the peak exists.

19. The mass spectrometer according to claim 13, wherein
the control unit is configured to, in the processing of determining, compare the degree of matching with a threshold, and determine that the component to be analyzed is present when the degree of matching is equal to or larger than the threshold.

20. The mass spectrometer according to claim 13, wherein
the control unit is configured to, in the determination, compare the degree of matching with a first threshold and a second threshold larger than the first threshold, determine that the component to be analyzed is absent when the degree of matching is smaller than the first threshold, and determine that the component to be analyzed is present when the degree of matching is equal to or larger than the second threshold.

21. The mass spectrometer according to claim 13, wherein
the control unit is configured to further execute:
processing of correcting a mass of the first mass spectrum in accordance with an ionization reaction in the preprocessing unit.

22. The mass spectrometer according to claim 13, wherein
the component to be analyzed is an organic chlorine compound.

23. The mass spectrometer according to claim 13, wherein
the component to be analyzed is an organic bromine compound.
